(19) United States
(12) Patent Application Publication
(10) Pub. No.: US 2025/0186513 A1
(43) Pub. Date: Jun. 12, 2025

(54) METHANOGENS AND PREVOTELLA AS MICROBIAL CONVENER FOR THE TREATMENT OF DISEASES AND DIAGNOSIS THEREOF

(71) Applicant: Cedars-Sinai Medical Center, Los Angeles, CA (US)

(72) Inventors: Mark Pimentel, Los Angeles, CA (US); Gabriela Leite, Porter Ranch, CA (US); Ruchi Mathur, Los Angeles, CA (US); Ali Rezaie, Beverly Hills, CA (US); Maria Villanueva, Los Angeles, CA (US)

(73) Assignee: Cedars-Sinai Medical Center, Los Angeles, CA (US)

(21) Appl. No.: 18/855,474

(22) PCT Filed: Apr. 18, 2023
(86) PCT No.: PCT/US2023/065909

§ 371 Date: Oct. 9, 2024

Related U.S. Application Data
(60) Provisional application No. 63/334,888, filed on Apr. 26, 2022.

(51) Int. Cl. A61K 35/74; A61P 1/00; C12Q 1/689

(52) U.S. Cl. CPC A61K 35/74; A61P 1/00; C12Q 1/689

(57) ABSTRACT

The invention provides for methods of detecting and improving microbial diversity in a subject's gastrointestinal system.

### CROSS-REFERENCE TO RELATED APPLICATIONS

This application includes a claim of priority to U.S. provisional patent application No. 63/334,888, filed Apr. 26, 2022, the entirety of which is hereby incorporated by reference.

### FIELD OF INVENTION

This invention relates to the detection regarding microbial diversity and methods of treating diseases and conditions relating to the lack of microbial diversity.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

We recently characterized the small intestinal bacterial microbiome and compared it to stool in humans for the first time. The study showed that the small bowel has a composition that is markedly different from the colon and may be important in human health and disease. A less understood component of the gut microbiota is the domain Archaea across the span of the gut.

Some members of the domain Archaea use hydrogen produced during microbial fermentation to create methane. Methane gas is known to slow intestinal transit. As a consequence, higher levels of methane and methanogens are found in constipated patients. Methane is also associated with other conditions, such as obesity. Thus, it is important to understand the distribution and composition of methanogens along the entire human luminal gastrointestinal tract which had not been fully characterized.

### SUMMARY OF THE INVENTION

The following embodiments and aspects thereof are described and illustrated in conjunction with compositions and methods which are meant to be exemplary and illustrative, not limiting in scope.

Various embodiments provide for a method of improving microbial diversity in a subject's gastrointestinal system, comprising: administering a microbial convener to the subject, the microbial convener comprising methanogen, Prevotella or both.

In various embodiments, the microbial convener can comprise both methanogens and Prevotella.

In various embodiments, the methanogen can be Methanobrevibacter, Methanomassiliicoccus luminyensis, Methanosphera stadtmaniae, Methanothermobacter, or combinations thereof. In various embodiments, the Methanobrevibacter can be M. smithii, M. ruminantium or both.

In various embodiments, the Methanothermobacter can be M. thermoautotrophicus, M. wolfeii, or both.

In various embodiments, the subject has been determined to have a low microbial diversity.

In various embodiments, the low microbial diversity can comprise the subject having low levels of Methanobrevibacter, Methanomassiliicoccus luminyensis, Methanosphera stadtmaniae, Methanothermobacter or combinations thereof as compared to each microorganism's reference value.

In various embodiments, the Prevotella can be Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella maculosa, Prevotella marshii, Prevotella melaninogenica, Prevotella micans, Prevotella multiformis, Prevotella oralis, Prevotella oris, Prevotella oulorum, Prevotella pallens, Prevotella salivae, Prevotella stercorea, Prevotella timonensis, Prevotella veroralis or combinations thereof.

In various embodiments, the method can further comprise determining the microbial diversity from a gastrointestinal sample obtained from the subject before administering the microbial convener. In various embodiments, determining the microbial diversity can comprise detection of low levels of Methanobrevibacter in the gastrointestinal sample as compared to a reference value. In various embodiments, determining the microbial diversity can comprise detection of low levels of Methanobrevibacter, Methanomassiliicoccus luminyensis, Methanosphera stadtmaniae, Methanothermobacter or combinations thereof as compared to each microorganism's reference value. In various embodiments, determining the microbial diversity can comprise detection of low levels of Prevotella.

In various embodiments, the subject can have one or more symptoms of inflammatory bowel disease (IBD). In various embodiments, the IBD can be Crohn's disease or ulcerative colitis.

In various embodiments, the subject can have one or more symptoms of an endocrine disorder. In various embodiments, the endocrine disorder can be polycystic ovary syndrome (PCOS).

Various embodiments provide for a method of detecting low microbial diversity in a subject in need thereof, comprising: assaying a biological sample, obtained from the subject's gastrointestinal system, to determine methanogen levels, Prevotella levels, or both; comparing the methanogen levels to a methanogen reference value, comparing Prevotella levels to a Prevotella reference value, or both; and detecting that the subject has low microbial diversity when the methanogen levels, Prevotella levels, or both are lower than each reference value.

In various embodiments, the subject can have one or more symptoms of inflammatory bowel disease.

In various embodiments, the subject can have one or more symptoms of endocrine disorder.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts abundance of the most prevalent archaeal families in small bowel segments and stool.

FIG. 2 depicts abundance of the most prevalent archaeal genera among the small bowel segments and stool.

FIG. 3 depicts correlations between relative abundance of phylum Bacteroidetes in the duodenal microbiome and duodenal microbial diversity.

FIG. 4 shows that healthier microbiome is associated with increased abundance of Prevotella. Women not receiving hormone replacement therapy (HT−), had less abundances of Prevotella in the small bowel as compared to women receiving hormone replacement therapy (HT+), which presented similar levels when compared to younger women at reproductive age (RA).

FIG. 5 depicts association between the family Prevotellaceae and small bowel microbial diversity measured by Simpsons, Shannon and Chao indices. Prevotella is the most important representative of this family in the small bowel (see next figure-FIG. 6).

FIG. 6 shows that Prevotella is the most important representative of the family Prevotellaceae in the small bowel (circle).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Treatment” and “treating,” as used herein refer to both therapeutic treatment and prophylactic or preventative measures, wherein the object is to prevent, slow down and/or lessen the disease even if the treatment is ultimately unsuccessful.

As used herein, the term “administering,” refers to the placement an agent as disclosed herein into a subject by a method or route which results in at least partial localization of the agents at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions may be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions may be in the form of aerosol, lotion, cream, gel, ointment, suspensions, solutions or emulsions. In accordance with the present disclosure, “administering” may be self-administering. For example, it is considered as “administering” that a subject consumes a composition as disclosed herein.

The term “biological sample” as used herein denotes a sample taken or isolated from a biological organism. Exemplary biological samples include, but are not limited to, cheek swab, mucus, whole blood, blood, serum, plasma, urine, stool, intestinal aspirate, duodenal aspirate, stomach aspirate, saliva, semen, lymph, fecal extract, sputum, other body fluid or biofluid, cell sample, and tissue sample etc. The term also includes a mixture of the above-mentioned samples. The term “sample” also includes untreated or pretreated (or pre-processed) biological samples. The term “gastrointestinal sample” as used herein refers to a biological sample obtained from the gastrointestinal track.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal may be a human, non-human primate, mouse, rat, dog, cat, horse, or cow, but are not limited to these examples. In addition, the methods described herein may be used to treat domesticated animals and/or pets. In various embodiments, the subject is a human.

Diversity is important in the microbiome. Most conditions that cause GI disease (e.g. Crohn's disease or ulcerative colitis) are associated with reduced diversity. Certain microbes are characterized as disrupters which mean when they are abundant, they reduce the normal bacteria types and levels. Herein, we describe bacteria that are conveners such that they increase the abundance and levels of normal bacteria.

Normal bacteria are part of the core small intestinal microbiome (bacterial genera present in at least 90% of all subjects and that demonstrate stable or predictable dynamics over time). These are mostly: Streptococcus (about 37% of the small bowel microbiome), Veillonella (about 13% of the small bowel microbiome), Rothia (about 4% of the small bowel microbiome), Actinomyces (about 0.03% of the microbiome) and Granulicatella (about 0.03% of the small bowel microbiome). Prevotella ranges from 0.005 to 0.04% of the “normal” small bowel microbiome, thus, relative amounts below 0.005% of the total small bowel microbiome can be considered potentially abnormal.

Certain conditions reduced diversity in the gut and can cause diseases and condition. The diseases and conditions include but are not limited to Crohn's Disease, ulcerative colitis, microscopic colitis, and antibiotic associated diarrhea.

As discussed herein, methanogens more commonly colonize the small bowel. 50% of subjects only had archaea in the small bowel and had no detectable archaea in stool. The small bowel luminal archaeome is composed primarily of the genera Methanobrevibacter, Methanosphaera and Methanomassiliicoccus. The distribution of the most prevalent archaeal genera was different between individual small bowel segments, and also different in stool.

As described herein, adding a microbe that enhances the abundance and levels normal bacteria, symptoms from these diseases and conditions can be improved.

Various embodiments of the present invention provide for a method of improving microbial diversity in a subject's gastrointestinal system, comprising: administering a microbial convener to the subject, the microbial convener comprising methanogen, Prevotella or both.

Various embodiments of the present invention provide for a method of treating a subject having a lack of microbial diversity in a subject's gastrointestinal system, comprising: administering a microbial convener to the subject, the microbial convener comprising a methanogen, Prevotella or both.

Administering the microbial convener can be for a period of about 7 days. Additional examples include periods of about 5 days, 10 days, 14 days, and 21 days. Further examples include administering for two or more cycles of the microbial convener. For example, the subject is administered the microbial convener for a cycle of about 7 days and 7 days off, and then administered for another cycle of about 7 days. These cycles are not limited to 7 days each, for example, they can be about 5 days, about 10 days, about 14 days, or about 21 days. The rest period between administrations can be about 3 days, about 5 days, about 10 days, about 14 days or about 21 days.

In various embodiments, the microbial convener comprises a methanogen. In various embodiments, the microbial convener comprises Prevotella. In various embodiments, the microbial convener comprises both methanogen and Prevotella.

In various embodiments, the methanogen is Methanobrevibacter. Methanomassiliicoccus luminyensis, Methanosphera stadtmaniae, Methanothermobacter or combinations thereof.

In various embodiments, the Methanobrevibacter is M. smithii. In various embodiments, the Methanobrevibacter is M. ruminantium. In various embodiments, the Methanobrevibacter is both M. smithii and M. ruminantium.

In various embodiments, the Methanothermobacter is M. thermoautotrophicus, M. wolfeii, or both.

Dosages of methanogens that are provided or administered can be about 10⁹ to 10¹⁰ CFU per dose. Additional examples include but are not limited to 10⁸, 10⁹, 10¹⁰, 5⁸, 5⁹, 5¹⁰ CFR per dose.

In various embodiments, the Prevotella is Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella maculosa, Prevotella marshii, Prevotella melaninogenica, Prevotella micans, Prevotella multiformis, Prevotella oralis, Prevotella oris, Prevotella oulorum, Prevotella pallens, Prevotella salivae, Prevotella stercorea, Prevotella timonensis, Prevotella veroralis or combinations thereof. Particularly, Prevotella histicola, Prevotella oralis, Prevotella veroralis, Prevotella buccae, or combinations thereof can be beneficial.

Dosages of Prevotella that are provided or administered can be about 10⁹ to 10¹⁰ CFU per dose. Additional examples include but are not limited to 10⁸, 10⁹, 10¹⁰, 5⁸, 5⁹, 5¹⁰ CFR per dose.

In various embodiments, the subject has been determined to have a low microbial diversity in the gastrointestinal track. In various embodiments, the low microbial diversity comprises the subject having low levels of Methanobrevibacter, Methanomassiliicoccus luminyensis, Methanosphera stadtmaniae, Methanothermobacter, or combinations thereof as compared to each microorganism's reference value. In various embodiments, the low microbial diversity comprises the subject having low levels of Prevotella as compared to a reference value for Prevotella. In various embodiments, the low microbial diversity comprises the subject having low levels of both Methanobrevibacter and Prevotella as compared to each of their reference values. In various embodiments, the low microbial diversity comprises the subject having low levels of Methanobrevibacter, Methanomassiliicoccus luminyensis, Methanosphera stadtmaniae, Methanothermobacter, Prevotella or combinations thereof. In various embodiments, the low microbial diversity comprises the subject having low levels of two or more of Methanobrevibacter, Methanomassiliicoccus luminyensis, Methanosphera stadtmaniae, Methanothermobacter or Prevotella.

The reference value can depend on the type of condition that is related to the lack of microbial diversity. Different types of conditions may have a different reference values. In some embodiments, the reference value can be established from biological samples from a healthy subject. For example, if the biological sample is stool, then the reference value can be obtained from the stools of a healthy subject; that is, a subject who does not have low microbial diversity in the gut. In another example, if the biological sample or gastrointestinal sample is small intestinal aspirate, the reference value can be obtained from the small intestinal aspirate of a healthy subject. In other embodiments, the reference value is the average microorganism count for the same type of biological sample from a population of healthy subjects. In some embodiments, the population of healthy subjects can range from at least three healthy individuals to 25 healthy individuals, from at least 26 healthy individuals to 50 healthy individuals, from at least 51 healthy individuals to 100 healthy individuals, and even more than 100 healthy individuals.

Non-limiting examples of low levels of Prevotella are levels of Prevotella below 0.005% of the total small bowel microbiome. In other embodiments, low levels of Prevotella are levels of Prevotella below 0.004%, 0.003%, 0.002%, or 0.001% of the total small bowel microbiome.

Non-limiting examples of low levels of Methanobrevibacter are levels of Methanobrevibacter below 1% of the total small bowel microbiome. In other embodiments, low levels of Methanobrevibacter are levels of Methanobrevibacter below 0.9%, 0.8%, 0.7%, 0.6%, 0.5%, 0.4%, 0.3%, 0.2% or 0.1% of the total small bowel microbiome. These percentages are based on stool analysis and thus, a different methodology used for quantification can generate a different reference value, which would be matched as discussed above.

In various embodiments, the microbial convener is released into the gastrointestinal system of the subject. For example, the microbial convener can be enteric-coated such that all or a substantial amount of the agent passes through the stomach and is released into the small intestine.

In various embodiments, the method further comprises determining the microbial diversity from a gastrointestinal sample obtained from the subject before administering the microbial convener. In various embodiments, the gastrointestinal sample is a small intestinal aspirate. In various embodiments, the gastrointestinal sample is stool.

In various embodiments, determining the microbial diversity comprises detection of low levels of Methanobrevibacter as compared to its reference value from a gastrointestinal sample. In various embodiments, determining the microbial diversity comprises detection of low levels of Methanobrevibacter, Methanomassiliicoccus luminyensis, Methanosphera stadtmaniae, Methanothermobacter, or combinations thereof as compared to each microorganism's reference value from a gastrointestinal sample. In various embodiments, determining the microbial diversity comprises detection of low levels of Prevotella as compared to a reference value for Prevotella from a gastrointestinal sample. In various embodiments, determining the microbial diversity comprises detection of low levels of both Methanobrevibacter and Prevotella as compared to each microorganism's reference value from a gastrointestinal sample. In various embodiments, determining the microbial diversity comprises detection of low levels of Methanobrevibacter, Methanomassiliicoccus luminyensis, Methanosphera stadtmaniae, Methanothermobacter Prevotella or combinations thereof as compared to each microorganism's reference value from a gastrointestinal sample. In various embodiments, determining the microbial diversity comprises detection of low levels of two or more of Methanobrevibacter. Methanomassiliicoccus luminyensis, Methanosphera stadtmaniae, Methanothermobacter and Prevotella as compared to each microorganism's reference value from a gastrointestinal sample.

In various embodiments, the subject has one or more symptoms of inflammatory bowel disease (IBD). In various embodiments, the subject has two or more symptoms of IBD. In various embodiments, the subject has three or more symptoms of IBD. Symptoms of IBD include but are not limited to persistent diarrhea, abdominal pain, rectal bleeding/bloody stools, weight loss, and fatigue. In various embodiments, the IBD is Crohn's disease or ulcerative colitis (UC). In these embodiments, administering the microbial convener improves one or more of the symptoms of IBD.

In various embodiments, if the subject has one or more symptoms of IBD (e.g., Crohn's disease or UC), two or more of Prevotella, Methanomassiliicoccus luminyensis, Methanosphera stadtmaniae, or Methanothermobacter is administered to the subject. In various embodiments, if the subject has one or more symptoms of IBD (e.g., Crohn's disease or UC), both Prevotella and Methanobrevibacter is administered to the subject.

In various embodiments, the subject has one or more symptoms of antibiotic associated diarrhea. In various embodiments, the subject has one or more symptoms of antibiotic associated diarrhea and both Prevotella and Methanobrevibacter is administered to the subject. In various embodiments, the subject has one or more symptoms of antibiotic associated diarrhea and two or more of Prevotella, Methanomassiliicoccus luminyensis, Methanosphera stadtmaniae, or Methanothermobacter is administered to the subject. In these embodiments, administering the microbial convener reduces the frequency or amount of diarrhea in the subject.

In various embodiments, the subject has one or more symptoms of an endocrine disorder. A nonlimiting example of an endocrine disorder is polycystic ovary syndrome (PCOS).

In various embodiments, if the subject has one or more symptoms of an endocrine disorder (e.g., PCOS), Prevotella is the microbial convener administered to the subject. In various embodiments, if the subject one or more symptoms of an endocrine disorder (e.g., PCOS), both Prevotella and Methanobrevibacter. In various embodiments, if the subject one or more symptoms of an endocrine disorder (e.g., PCOS), Prevotella, Methanomassiliicoccus luminyensis, Methanosphera stadtmaniae, Methanothermobacter or combinations thereof is administered to the subject. In these embodiments, administering the microbial convener improves one or more of the symptoms of the endocrine disorder.

Various embodiments of the present invention provide for a method of detecting low microbial diversity, comprising: assaying a biological sample, obtained from a subject's gastrointestinal system, to determine methanogen levels, Prevotella levels, or both; comparing the methanogen levels to a methanogen reference value, comparing Prevotella levels to a Prevotella reference value, or both; and detecting that the subject has low microbial diversity when the methanogen levels, Prevotella levels, or both are lower than each reference value.

In various embodiments, the method further comprising administering a microbial convener to the subject, the microbial convener comprising methanogen, Prevotella or both. The methanogen can be those as discussed here, and the Prevotella can also be those as discussed herein.

Administering the microbial convener can be for a period of about 7 days. Additional examples include periods of about 5 days, 10 days, 14 days, and 21 days. Further examples include administering for two or more cycles of the microbial convener. For example, the subject is administered the microbial convener for a cycle of about 7 days and 7 days off, and then administered for another cycle of about 7 days. These cycles are not limited to 7 days each; for example, they can be about 5 days, about 10 days, about 14 days, or about 21 days. The rest period between administrations can be about 3 days, about 5 days, about 10 days, about 14 days or about 21 days.

In various embodiments, detecting the various levels of microorganism that are present in the subject's gastrointestinal system comprises using a technique selected from the group consisting of PCR, DNA sequencing to determine the presence of each microorganism, culturing for each microorganism, 16S rRNA sequencing, and combinations thereof. Examples of DNA sequencing include but are not limited to Sanger sequencing, shotgun sequencing, and high-throughput sequencing (e.g., next-generation “short-read” and third-generation “long-read” sequencing methods (e.g., single molecule real time (SMRT) sequencing, nanopore DNA sequencing).

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Comprehensive Characterization of the Gastrointestinal Luminal Archaeome: Data from the REIMAGINE Study

### Methods

Subjects aged 18-85 years undergoing esophagogastroduodenoscopy (EGD) or double balloon enteroscopy (DBE) without bowel preparation were recruited for the REIMAGINE study. All subjects included in this study also provided stool samples. Among subjects undergoing DBE, aspirates from the duodenum, jejunum, and farthest distance (FD) reached during DBE, were obtained using a sterile novel aspiration catheter. Stool samples were self-collected using the OMNIgene-GUT for Microbiome kit.

DNA from aspirates and stool was isolated using the MagAttract PowerSoil DNA Kit using the KingFisher Duo Prime System (Thermo Fisher Scientific). To detect Archaea, sequencing was performed from nested PCR amplicons generated using a combination of two primer pairs 344F-1041R/519F-806R previously published by Pausan et al. (2019) with some modifications. Pair-end (2×300) sequencing was carried out on a MiSeq platform (Illumina). Reference-based Operational Taxonomic Unit (OTU) clustering was performed using the SILVA v132 database.

### Results

Samples from 14 subjects with matching stool samples (age=52.7±14.2, male=8) were included for analysis. Interestingly, all subjects had detectable Archaea in the luminal contents of the small bowel (Table 1). In seven (50% of individuals), Archaea were only present in the small bowel and not in the stool.

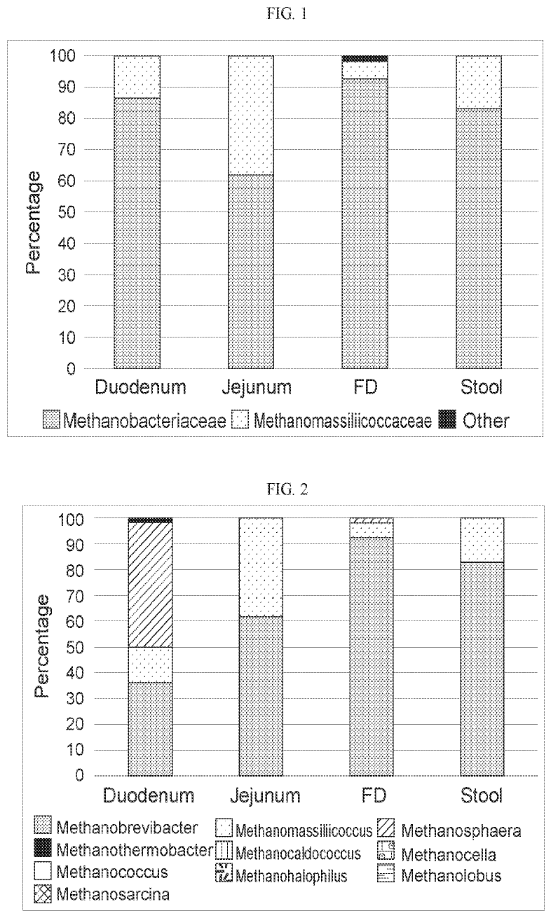

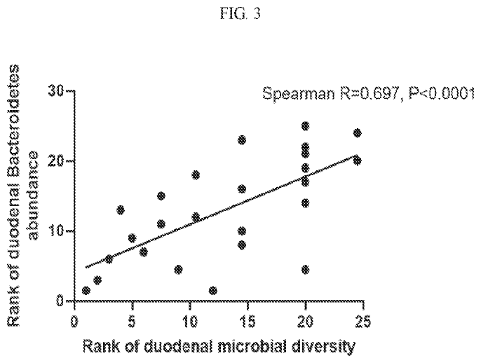

The most common archaeal families present in the small bowel or stool samples were Methanobacteriaceae and Methanomassiliicoccaceae. The family Methanobacteriaceae was the most prevalent in all small intestinal segments and stool (FIG. 1-2). The most abundant archaeal genera were Methanobrevibacter, Methanosphaera, and Methanomassiliicoccus. Methanobrevibacter was present in the small bowel of 100% of subjects while Methanomassiliicoccus was present in 85.7%. However, the prevalence of these three archaeal genera was different among the individual small bowel segments and was also different in stool (FIG. 2). While stool had a more diverse archaeal community (Table 1), the small bowel was more commonly colonized with the top three archaeal genera (FIG. 2). Methanosphaera was more abundant in the duodenum, whereas Methanobrevibacter was the most abundant in the jejunum and FD (FIG. 2).

TABLE 1
Archaeal genera found in small bowel and stool

| | Small Bowl | | Stool | |
|---|---|---|---|---|
| | | Number of | | Number of |
| Archael genera | Presence | individuals | Presence | individuals |
| Methanobrevibacter | Yes | 14/14 | Yes | 5/14 |
| Methanomassiliicoccus | Yes | 12/14 | Yes | 4/14 |
| Methonosphaera | Yes | 3/14 | Yes | 2/14 |
| Methanothermobacter | Yes | 1/14 | Yes | 1/14 |
| Methanocaldococcus | No | 0/14 | Yes | 1/14 |
| Methanocella | No | 0/14 | Yes | 1/14 |
| Methanococcus | No | 0/14 | Yes | 1/14 |
| Methanoha/ophilus | No | 0/14 | Yes | 1/14 |
| Methanolobus | No | 0/14 | Yes | 1/14 |
| Methanosarcina | No | 0/14 | Yes | 1/14 |

### Example 2

Female participants undergoing esophagogastroduodenoscopy who were postmenopausal and taking HT (HT+), postmenopausal but not taking HT (HT−), or of reproductive age and not taking exogenous hormones (RA), were identified and matched for body mass index (±3 kg/m²). DNAs were isolated from duodenal aspirates obtained during upper endoscopy. V3 and V4 libraries were used for 16S rRNA sequencing. Serum hormone levels were analyzed by Luminex FlexMap.

One of the most important taxa from phylum Bacteroidetes that contributed to these associations was the genus Prevotella, which also correlated with duodenal microbial diversity in postmenopausal women (Spearman R=0.658, P<0.0001).

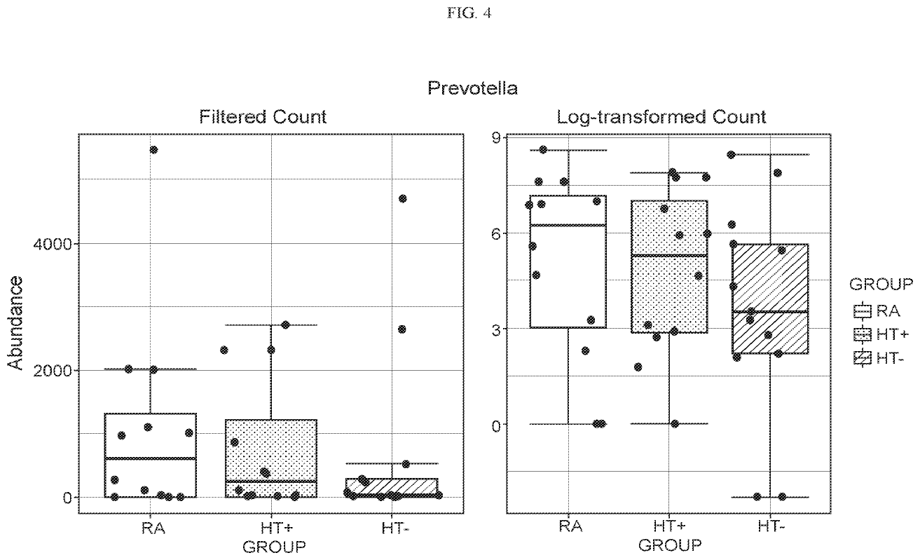

The relative abundance of genus Prevotella was decreased in the duodenum of the HT− group, and was completely depleted in two women from this group. In contrast, the relative abundance of Prevotella was slightly increased in the HT+ group, and was closer to that seen in the duodenum of the RA group (FIG. 4).

Across the postmenopausal groups (i.e., in both the HT− and HT+ groups), decreased duodenal diversity, increased fasting glucose levels, and decreased serum testosterone levels, were associated with decreased relative abundance of the phylum Bacteroidetes in the duodenal microbiome. Changes in the relative abundance of Bacteroidetes were largely due to the genus Prevotella, which was significantly reduced in the duodenal microbiome of the HT− group. In contrast, the relative abundance of Bacteroidetes and Prevotella was more comparable in the HT+ and RA groups.

### Example 3

In a cross-sectional study, current smokers (CS, n=24) were identified and matched to never-smokers (NS, n=27) and ex-smokers (XS, n=27) by age (+5 years), body mass index (BMI, +3), and sex. Current antibiotic users were excluded. The duodenal luminal microbiome was analysed in 1 aspirate sample per subject by 16S rRNA gene sequencing.

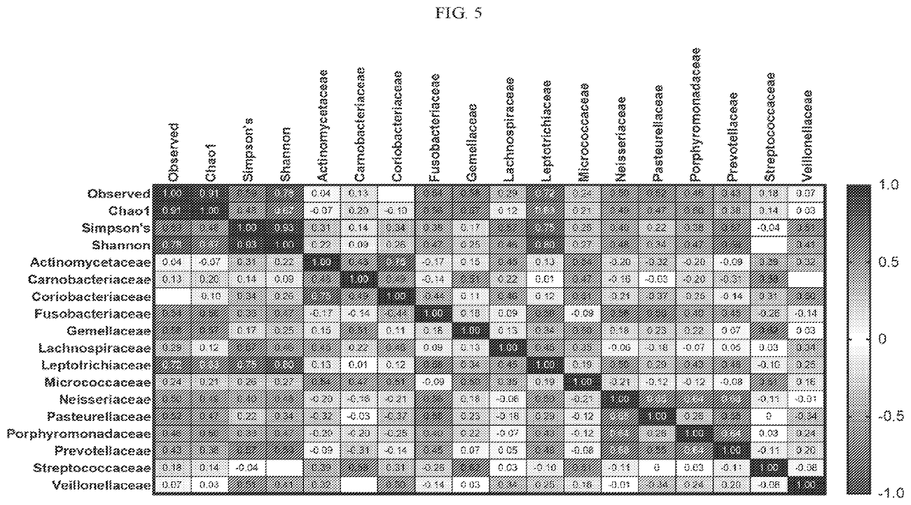

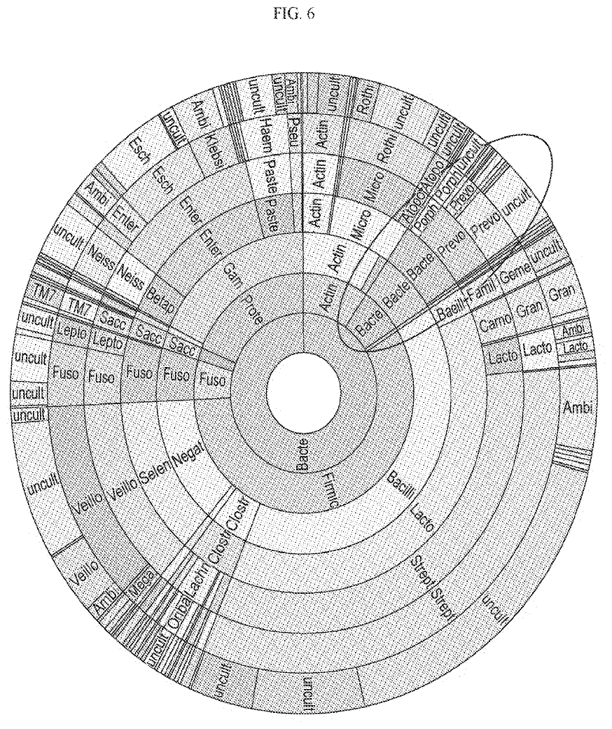

The prevalence of certain duodenal families was found to be positively associated with microbial diversity, including Prevotellaceae (phylum Bacteroidetes), Neisseriaceae (phylum Proteobacteria) and Porphyromonadaceae (phylum Bacteroidetes) (FIG. 5).

Relative abundances (RA) of families associated with increased duodenal microbial diversity, such as Prevotellaceae, are significantly lower in the small bowel of subjects that smoke (CS) when compared to non-smokers (NS). This is driven by lower RA of the genus Prevotella.

### Example 4

A patient presents with symptoms of IBD. The patient is tested for their methanogen levels and the results indicate a low level of methanogens. The patient is prescribed/administered a composition comprising Methanobrevibacter for about 7 days. The patient's IBD symptoms improved after the therapy.

### Example 5

A patient presents with symptoms of PCOS. The patient is tested for their levels of Prevotella. The results show that the patient has low levels of Prevotella. The patient is administered a composition comprising Prevotella for about 14 days. The patient's symptoms improved after the therapy.

### Example 6

A patient was previously tested for levels of Prevotella and methanogens present in their small bowel and was found to have low levels of both. The patient is administered a composition comprising Prevotella and Methanobrevibacter for about 10 days. The patient's small intestinal microbial diversity increased after the therapy.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of improving microbial diversity in a subject's gastrointestinal system, comprising:
administering a microbial convener to the subject, the microbial convener comprising methanogen, Prevotella or both.

2. The method of claim 1, wherein the microbial convener comprises both methanogens and Prevotella.

3. The method of claim 1, wherein the methanogen is Methanobrevibacter, Methanomassiliicoccus luminyensis, Methanosphera stadtmaniae, Methanothermobacter, or combinations thereof.

4. The method of claim 3, wherein the Methanobrevibacter is M. smithii, M. ruminantium or both.

5. The method of claim 3, wherein the Methanothermobacter is M. thermoautotrophicus, M. wolfeii, or both.

6. The method of claim 1, wherein the subject has been determined to have a low microbial diversity.

7. The method of claim 6, wherein the low microbial diversity comprises the subject having low levels of Methanobrevibacter, Methanomassiliicoccus luminyensis, Methanosphera stadtmaniae, Methanothermobacter or combinations thereof as compared to each microorganism's reference value.

8. The method of claim 1, wherein the Prevotella is Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella maculosa, Prevotella marshii, Prevotella melaninogenica, Prevotella micans, Prevotella multiformis, Prevotella oralis, Prevotella oris, Prevotella oulorum, Prevotella pallens, Prevotella salivae, Prevotella stercorea, Prevotella timonensis, Prevotella veroralis or combinations thereof.

9. The method of claim 1, further comprising determining the microbial diversity from a gastrointestinal sample obtained from the subject before administering the microbial convener.

10. The method of claim 9, wherein determining the microbial diversity comprises detection of low levels of Methanobrevibacter in the gastrointestinal sample as compared to a reference value.

11. The method of claim 9, wherein determining the microbial diversity comprises detection of low levels of Methanobrevibacter, Methanomassiliicoccus luminyensis, Methanosphera stadtmaniae, Methanothermobacter or combinations thereof as compared to each microorganism's reference value.

12. The method of claim 9, wherein determining the microbial diversity comprises detection of low levels of Prevotella.

13. The method of claim 1, wherein the subject has one or more symptoms of inflammatory bowel disease (IBD).

14. The method of claim 13, wherein the IBD is Crohn's disease or ulcerative colitis.

15. The method of claim 1, wherein the subject has one or more symptoms of an endocrine disorder.

16. The method of claim 15, wherein the endocrine disorder is polycystic ovary syndrome (PCOS).

17. A method of detecting low microbial diversity in a subject in need thereof, comprising:
assaying a biological sample, obtained from the subject's gastrointestinal system, to determine methanogen levels, Prevotella levels, or both;
comparing the methanogen levels to a methanogen reference value, comparing Prevotella levels to a Prevotella reference value, or both; and
detecting that the subject has low microbial diversity when the methanogen levels, Prevotella levels, or both are lower than each reference value.

18. The method of claim 17, wherein the subject has one or more symptoms of inflammatory bowel disease.

19. The method of claim 17, wherein the subject has one or more symptoms of endocrine disorder.
